(19) United States
(12) Patent Application Publication
(10) Pub. No.: US 2006/0052437 A1
(43) Pub. Date: Mar. 9, 2006

(54) Combination of an hmg-coa reductase inhibitor and a nitrate ester

(76) Inventor: Giovanni Scaramuzzino, Monza (IT)

Correspondence Address: SHOEMAKER AND MATTARE, LTD, 10 POST OFFICE ROAD - SUITE 110, SILVER SPRING, MD, 20910, US

(21) Appl. No.: 10/514,204

(22) PCT Filed: May 8, 2003
(86) PCT No.: PCT/EP03/04860

§ 371 Date: Nov. 12, 2004

(30) Foreign Application Priority Data
May 13, 2002 (IT) MI2002A001012

(51) Int. Cl. A61K 31/401; A61K 31/366; A61K 31/21; A61K 31/225

(52) U.S. Cl. 514/423; 514/548; 514/460; 514/509

(57) ABSTRACT

The present invention relates to the therapeutical combination of an HMG-CoA reductase inhibitor (statin) and a nitrate ester and is useful mainly for the preparation of medicaments for the prevention and treatment of coronary diseases as myocardial infarction and cerebrovascular diseases as stroke. Particularly, as a nitrate ester, a nitrate prodrug of aspirin, salicylic acid or vitamin E is used. Said compositions, compared to single components, have the advantages to be without toxic effects, mainly due to statins, and to be more effective.

The present invention relates to the therapeutical combination of an HMG-CoA reductase inhibitor (statin) and a nitrate ester and it is useful mainly for the preparation of medicaments for the prevention and treatment of coronary diseases like myocardial infarction and cerebrovascular diseases like stroke. Particularly, as a nitrate ester, a nitrate prodrug of aspirin, salicylic acid or vitamin E is used. Said compositions, compared to single components, have the advantages to be without toxic effects (mainly due to statins) and to be more effective.

Coronary diseases (myocardial infarction and other fatal coronary disesases) represent the most common cause of mortality in the most developed countries. Clinical complications of coronary diseases lead to substantial disability and are a major cause of the rising cost of health care.

Among the major risk factors for coronary diseases there are hypercholesterolemia, diabetes mellitus, hypertension and cigarette smoking.

The treatment with 3-hydroxy-3-methylglutaryl coenzyme A (HMG-CoA) reductase inhibitors, commonly referred to as statins, decreases the incidence of cardiovascular events and the mortality in patients with coronary diseases (Scandinavian Simvastatin Survival Study, Lancet, 1994, 344, 1383-1389) and in healthy population at high risk of coronary diseases (Shepherd J et al, N Eng J Med, 1995, 333, 1301-1307).

The statins currently sold all over the world are lovastatin, simvastatin, pravastatin, fluvastatin, atorvastatin, pitavastatin and in the near future rosuvastatin.

Such favorable effects of HMG-CoA reductase inhibitor drugs have been thought to be mediated through a decrease in serum low-density lipoprotein (LDL) cholesterol and triglycerides and an increase of serum high-density lipoprotein (HDL) cholesterol.

Despite said favorable effects, coronary diseases are very common. In industralized countries they represent the main cause of mortality and only in the United States of America, for example, they cause over 500,000 deaths a year (IMS-HEALTH).

Since at the beginning of atherosclerosis there are high plasmatic levels of cholesterol and lipids, said drugs should decrease the risk of atherosclerosis.

Arteriosclerosis is a series of modifications constituted by thickening and loss of elasticity of the wall of an arterial vessel. Both arteriosclerosis which damages the small arteries of the body and atherosclerosis, characterized by formation of atheromas in the muscular large and medium (coronary, carotid and femoral) arteries and in the elastic arteries as aorta, belong to this group. In the tunica intima of these arteries there are irregularly distributed lipidic deposits.

Indeed, despite many other clinical studies, besides that one said before, have revealed a significant decrease of coronary clinical events, most of said studies have showed only minimal degrees of regression of coronary atheromas, thereby suggesting, as to statins, several mechanisms of action which are cholesterol biosynthesis-independent and namely the stabilization of plaques, an improvement of endothelial functions, and a decreased thrombogenicity. Particularly, several evidences (Laufs U et al, Circulation, 1998, 97, 1129-1135) have proved that statins induce endothelial nitric oxide synthase (eNOS) and this can explain many beneficial activities of statins which are therefore not correlated with the inhibition of the HMG-CoA reductase.

Statins show several toxic effects, the most important of which are hepatic and muscular as well as metabolic toxicities.

As for hepatic toxicity (Maron D J et al, Circulation, 2000, 101(2), 207), in about 1% of patients the level of transaminases increase greater than 3-fold. The transaminases are glutamate oxaloacetate transaminase (or GOT or aspartate transferase or AST) and glutamate pyruvate transaminase (or GPT or alanine transferase or ALT). If this occurs, the drug should be stopped and the level of transaminases generally return to baseline within 2 to 3 months. Both baseline and periodic monitoring of liver transaminases are recommended.

As for metabolic toxicity (JAMA 2002, 287, 598-605), the treatment with simvastatin in man has been recently found to lead to an increase of 13% of insulin levels not modifying the glucose levels and thereby suggesting a decrease of the sensibility to insulin. This effect is potentially dangerous because the decreased sensibility to insulin leads to insulin resistance and then to diabetes mellitus which is, as said before, an important risk factor of coronary diseases, as well as renal injuries and blindness. Besides this effects on insulin, statins have other metabolic effects because they decrease up to 22% the concentration of important antioxidants such as alfa-tocopherol, beta-carotene and coenzyme Q-10. Also this is worrying because it has been known for many years the importance of the antioxidants in the protection from atherosclerosis, Alzheimer's disesase and tumors.

As for muscular toxicity (Maron D J et al, Circulation, 2000, 101(2), 207), myopathy is the most important toxic effect of statins and it is present in about 0.1% of patients. It is defined as muscle pain or weakness associated with creatine kinase (CK) levels higher than 10 times the upper limit of normal. If myopathy is not recognized and the drug is stopped, rhabdomyolysis may result which can lead to death due to acute renal failure.

The risk of myopathies is greater for cerivastatin. For this reason cerivastatin has been retired from the market in the month of August 2001, after 52 deaths which were attributable to rhabdomyolysis. To date such deaths exceed 100 units. The withdrawal of cerivastatin (also referred to as Lipobay or Baycol from its trade marks) from the market has raised much sensation and many doubts about the safety of all statins all over the world (Curr Control Trials Cardiovasc Med 2001, 2 (5), 205-207).

Therefore, currently there are not any statins in advanced clinical development all over the world, besides pitavastatin, marketed last year in Japan, and rosuvastatin, whose marketing has been so far delayed. This is significant because it happens despite, as said before, coronary diseases are the first cause of deaths in the Occident and despite statins are the second therapeutical class for annual sales all over the world (IMS HEALTH). In the last 6 years at least 16 clinical trials of statins have been stopped, most of which in the early development stages.

More effective and mainly more safe therapies for primary and secondary prevention and for the treatment of coronary diseases are therefore extremely necessary.

As said before, there is a great interest in possible cholesterol-independent mechanisms of action of statins and in particular in the induction of eNOS. Recently (Dobrucki L W et al, Med Sci Monit, 2001, 7(4), 622-627) statins have been proved to stimulate effectively, even if in a limited way, the nitric oxide release by eNOS which is present in the endothelium.

It has been known for over a decade that the loss of the endothelial production of nitric oxide damages endothelium-dependent dilatation and promotes vasospasm in the arteriosclerotic arteries. More recently eNOS disfunctions have been proved to promote the first stages of arteriosclerosis thanks to protective effects of nitric oxide against leukocytes recruitment, oxidative processes and migration and proliferation of smooth muscle cells (for a complete review see Napoli C et al, Nitric Oxide, 2001, 5(2), 88-97).

To exploit these properties of nitric oxide it has been tried the administration of L-arginine which is the substrate for eNOS to produce nitric oxide, or the insertion of eNOS gene also in man has been tried (Cable D G et al, Circulation, 1997, 96, II-8).

The use of nitric oxide donors, as for example nitrate esters, has been another approach.

In this field, among new therapeutical prospects, an aspirin prodrug, referred to as nitroaspirin or NCX-4016 which can release nitric oxide in vivo and is useful for the prevention of atherosclerosis, has been recently cited (Drug Dev. Res., 2001, 53, 237-243). NCX-4016, besides exploiting said nitric oxide properties for atherosclerosis, has excellent antiinflammatory and antithrombotic properties and, compared to aspirin, is not toxic at gastric level (Del Soldato P et al, 1999, Trends Pharmacol. Sci., 20, 319-323).

Said derivative would be potentially very useful for primary and secondary prevention of coronary events but, disadvantageously, it does not decrease in any way the levels of cholesterol and, on the contrary, it increases significantly total cholesterol and mainly LDL-cholesterol in the model of hypercholesterolemic rabbit described in the above-mentioned article.

It is important to point this out because, despite the importance of the HDL-cholesterol, the most recent guidelines in the United States of America within the “National Cholesterol Education Program” (JAMA, 2001, 285(19), 2486-2497) clearly show that the increased levels of LDL-cholesterol increase the risk of coronary diseases, that the therapies which decrease the LDL-cholesterol decrease the risk of coronary diseases and that this must be the primary target of hypocholesteremic therapies.

Generally, many nitrate esters of drugs or molecules having useful properties in this field would have the advantage, compared to the original molecules or drugs, to be able to exploit the above-said properties of nitric oxide.

For example, the nitrate prodrugs of vitamin E and salicylic acid, described in the European Patent Application no. 02425075.5 in the name of the same Applicant (see examples 2 and 15), are particularly useful for the prevention of atherosclerosis because they combine the properties of nitric oxide with those of vitamin E and salicylic acid.

However, also in this case, said derivatives disadvantageously do not decrease the levels of total cholesterol and LDL-cholesterol.

Compared to statins and the new therapeutical prospects like nitrate esters (e.g. NCX-4016), more effective and mainly more safe therapies for primary and secondary prevention and for the treatment of coronary diseases are therefore extremely necessary.

With great surprise, we have found the administration of the combination of an HMG-CoA reductase inhibitor and a nitrate prodrug of aspirin, salicylic acid or vitamin E to show an unexpected synergic effect and to be significantly more effective and with lower toxic effects in the prevention and treatment of coronary events compared to the components of the combination administered alone.

An object of the present invention is a pharmaceutical composition comprising:
a. an amount of an HMG-CoA reductase inhibitor or a pharmaceutically acceptable salt thereof
b. an amount of a nitrate ester or a pharmaceutically acceptable salt thereof
c. pharmaceutically acceptable excipients.

The HMG-CoA reductase inhibitor is preferably selected from the following compounds or pharmaceutically acceptable salts thereof (e.g. sodium salt, calcium salt): atorvastatin (HI), bervastatin (HIX), cerivastatin (HVIII), crilvastatin, dalvastatin, fluvastatin/fluindostatin (HVII), glenvastatin, lovastatin (HVI), mevastatin, pitavastatin (itavastatin, nisvastatin, NK-104) (HX), pravastatin (HII), rosuvastatin (S-4522/ZD-4522) (HIII), simvastatin (HV).

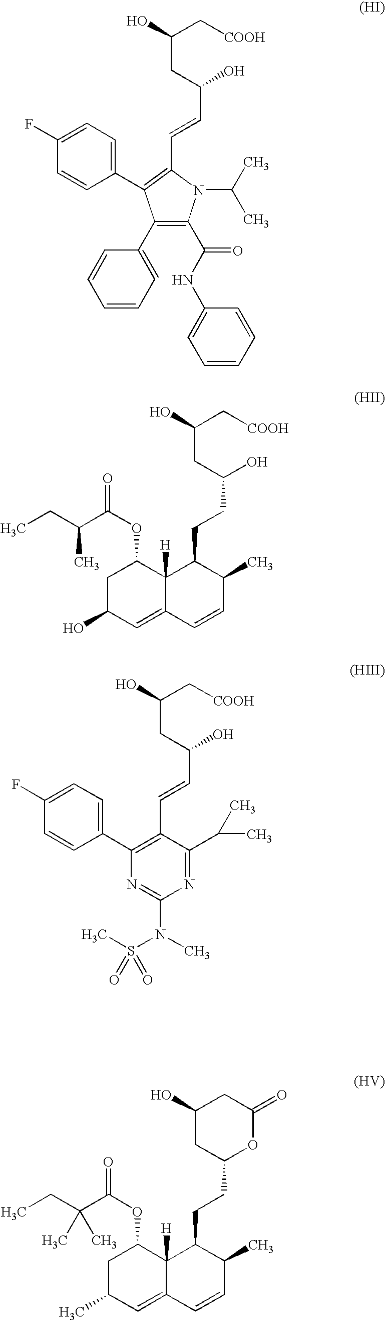

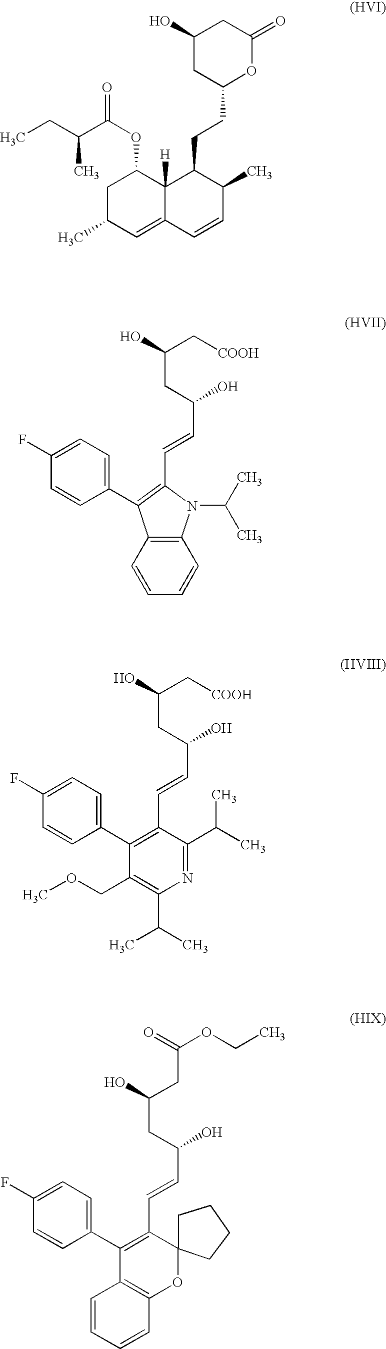

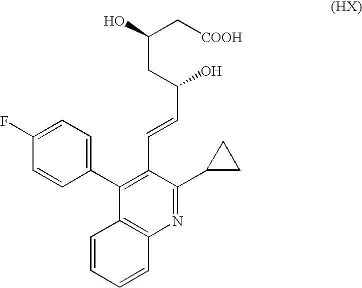

More preferably, the HMG-CoA reductase inhibitor is selected from: atorvastatin hemicalcium salt (HIa), fluvastatin/fluindostatin sodium salt (HVIIa), pitavastatin (itavastatin, nisvastatin, NK-104) hemicalcium salt (HXa), pravastatin sodium salt (HIIa), rosuvastatin hemicalcium salt (S-4522/ZD-4522) (HIIIa), simvastatin (HV).

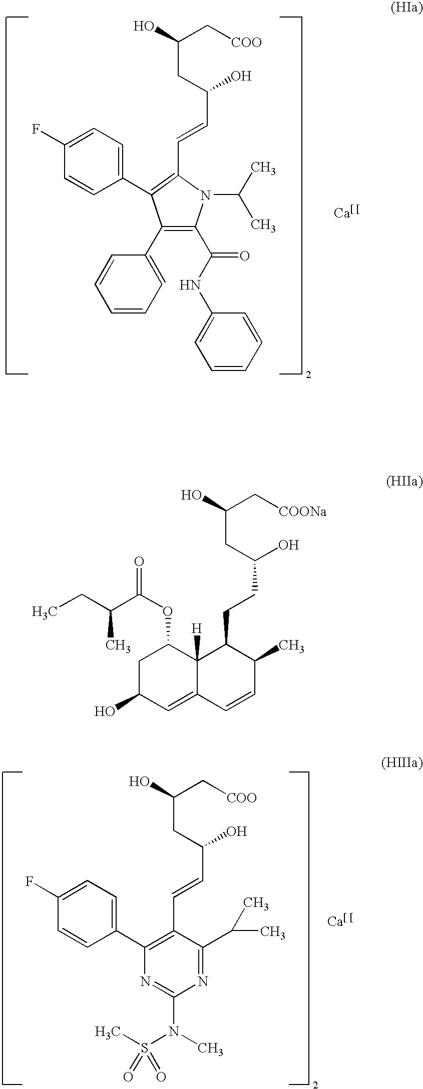

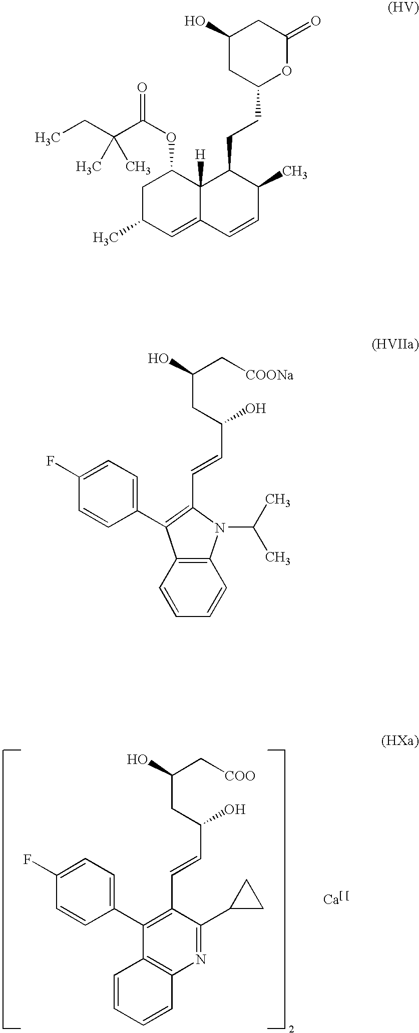

The nitrate ester is preferably a nitrate prodrug of aspirin/salicylic acid or vitamin E, respectively represented by general formulae (I) and (II) or pharmaceutically acceptable salts thereof:

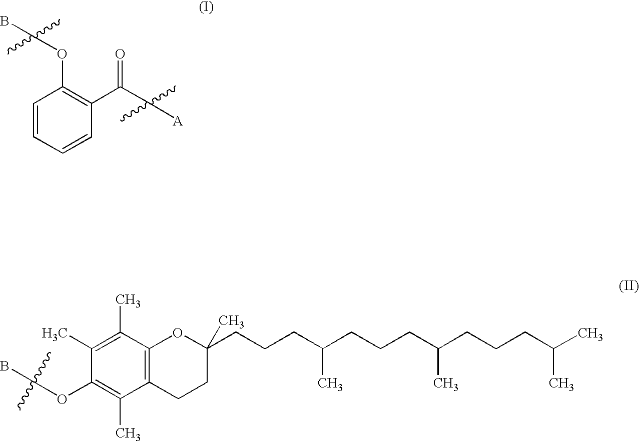

wherein:

A is selected from:

—O—R₂; —O—R₁—O—R₂; —O—R₁—S—R₂; —O—R₁—NR₂—R₂; —S—R₂; —S—R₁—O—R₂;

—S—R₁—S—R₂; —S—R₁—NR₂—R₂; —NR₂—R₂; —NR₂—R₁—O—R₂; —NR₂—R₁—S—R₂;

—NR₂—R₁—NR₂—R₂; —O—(CO)—R₂;

—O—R₁—ONO₂; —O—R₁—O—R₁—ONO₂; —O—R₁—S—R₁—ONO₂; —O—R₁—NR₂—R₁—ONO₂;

—S—R₁—ONO₂; —S—R₁—O—R₁—ONO₂; —S—R₁—S—R₁—ONO₂; —S—R₁—NR₂—R₁—ONO₂;

—NR₂—R₁—ONO₂; —NR₂—R₁—O—R₁—ONO₂; —NR₂—R₁—S—R₁—ONO₂;

—NR₂—R₁—NR₂—R₁—ONO₂; —O—(CO)—R₁—ONO₂;

—R₁— is a saturated or unsaturated, linear or branched alkylene having from 1 to 21 carbon atoms or a saturated or unsaturated, optionally heterosubstituted or branched cycloalkylene having from 3 to 7 carbon atoms or an optionally heterosubstituted arylene having from 3 to 7 carbon atoms;

—R₂ is —H or a saturated or unsaturated, linear or branched alkyl having from 1 to 21 carbon atoms or a saturated or unsaturated, optionally heterosubstituted or branched cycloalkyl having from 3 to 7 carbon atoms or an optionally heterosubstituted aryl having from 3 to 7 carbon atoms;

—R₁— and —R₂ may be substituted with —OH, —SH, —F, —Cl, —Br, —OPO₃H₂, —COOH, —NH₂ or with a saturated or unsaturated, linear or branched alkyl having from 1 to 10 carbon atoms or with a saturated or unsaturated, optionally heterosubstituted or branched cycloalkyl having from 3 to 7 carbon atoms;

B is selected from:

—R₂; —(CO)—R₂; R₁—O—R₂; —PO(O—)O—R₂; —ONO₂; —(CO)—R₁—ONO₂;

—(CO)—R₁—O—R₁—ONO₂; —(CO)—R₁—S—R₁—ONO₂;

—(CO)—R₁—NR₂—R₁—ONO₂; R₁—O—R₁—ONO₂

in the general formula (I) the derivatives wherein both -A and —B simultaneously do not contain any —ONO₂ group are excluded;

the bond indicated by the undulated line is hydrolyzable in vivo by metabolic or enzymatic activity and in particular is a carboxylic ester, a glycoside, an acetal, a ketal, a phosphoric ester, a phosphonic ester, a sulphonic ester;

the pharmaceutically acceptable salts are salts from inorganic acids (e.g. nitrate, nitrite, hydrochloride, hydrobromide, sulphate, phosphate), salts from organic acids (e.g. citrate, tartrate, acetate, maleate, fumarate, oxalate, p-toluensulphonate, methanesulphonate, ethanesuphonate, benzenesulphonate), from inorganic bases (e.g. ammonium, sodium, lithium, potassium, magnesium, calcium and zinc salts) or from organic bases (e.g. ammonium salts of organic amines). Nitrate salts, hydrochloride salts, sodium salts and potassium salts are preferred. Nitrate salts are more preferred.

Preferably in the general formula (I):

A is selected from —OH and the following monovalent radicals:

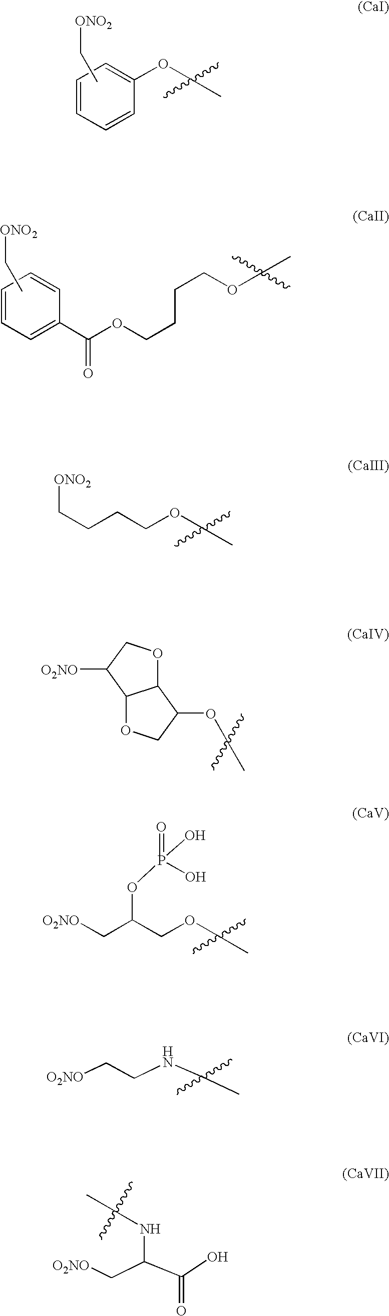

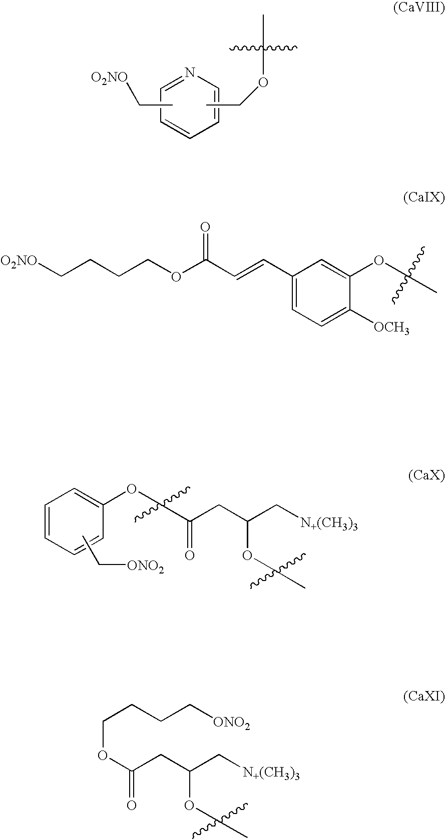

—B is selected from —H , —(CO)CH₃ and the following monovalent radicals:

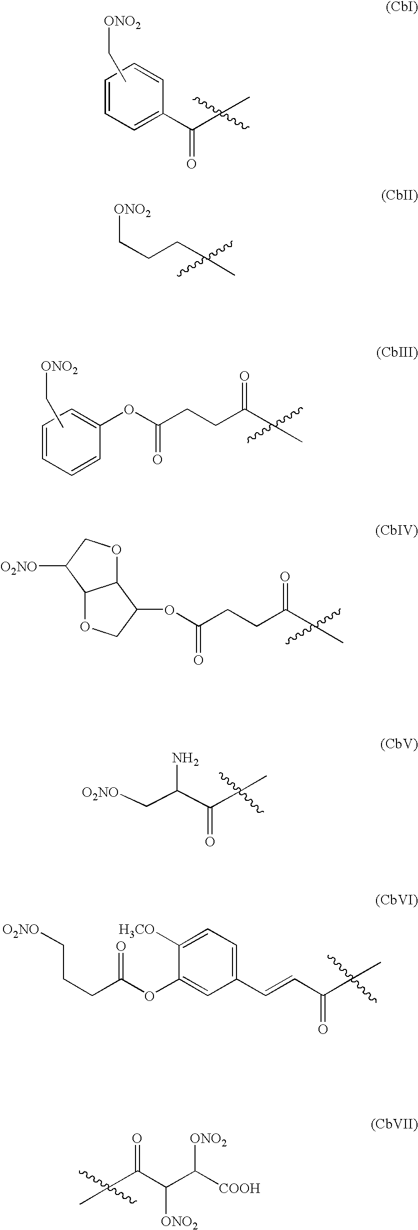

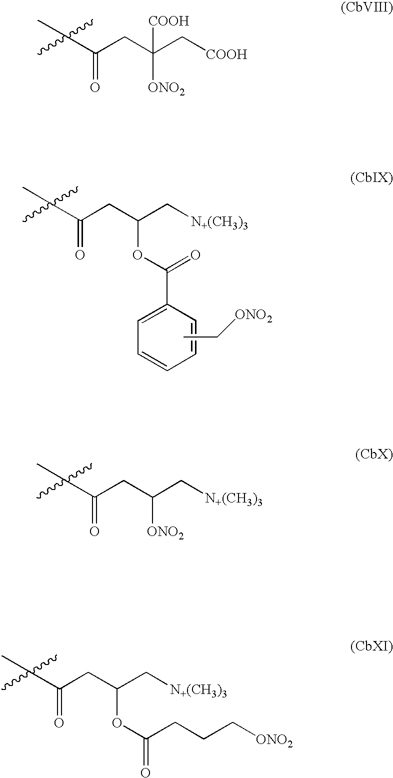

wherein the bond indicated by the undulated line may be a carboxylic ester or a carboxylic amide and wherein in the general formula (I) A may not be —OH when —B is —H or acetyl.

Preferably, the nitrate prodrug of aspirin/salicylic acid or vitamin E is selected from the following molecules or pharmaceutically acceptable salts thereof:

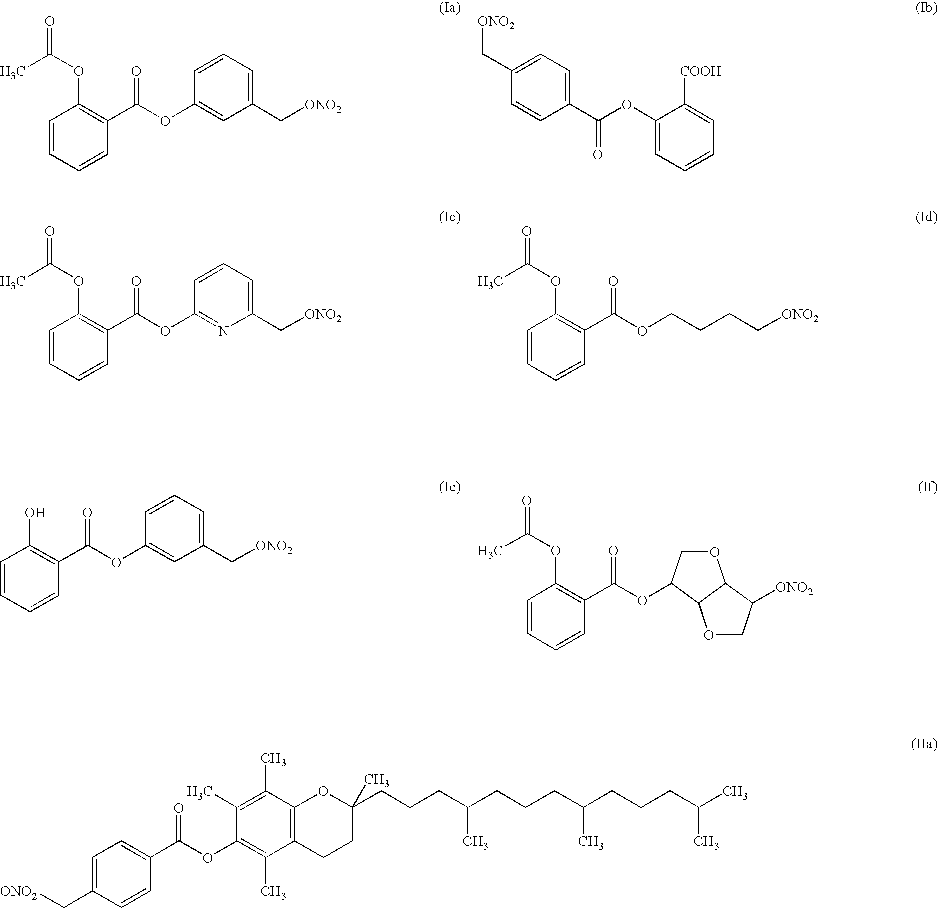

Even more preferably the nitrate prodrug of aspirin is 2-acetyloxy-benzoic acid 3-nitroxymethyl-phenyl ester (Ia).

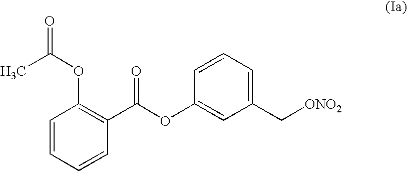

The statins of the invention are purchased from commercial catalogs or synthesized according to the literature. Particularly lovastatin is purchased from Sigma-Aldrich catalog (code M2147), pravastatin sodium salt is purchased from Calbiochem catalog (code 524403) or synthesized according to U.S. Pat. No. 4,346,227, simvastatin is purchased from Calbiochem catalog (code 567020) or synthesized according to WO9965892, atorvastatin hemicalcium salt is purchased from Calbiochem catalog (code 189290) or synthesized according to EP409281, fluvastatin sodium salt is purchased from Calbiochem catalog (code 3440959) or synthesized according to WO8402131, rosuvastatin hemicalcium salt is synthesized according to EP521471 and pitavastatin hemicalcium salt is synthesized according to Bioorg Med Chem Lett 1999, 9(20), p. 2977.

2-Acetyloxy-benzoic acid 3-nitroxymethyl-phenyl ester (Ia) is synthesized according to EP871606.

The nitrate esters of the invention and in particular the nitrate prodrugs of aspirin, salicylic acid and vitamin E of general formulae (I) and (II) are synthesized according to known reactions described in literature (see for example “Advanced Organic Chemistry” of J. March, Wiley Interscience, IV ed.).

The compounds of this invention can be synthetized according to known techniques and in particular we can distinguish:
A) methods for the synthesis of nitroxy substituted linkers
B) methods for the synthesis of in vivo hydrolyzable bonds
C) methods for the synthesis of nitroxy substituted organic esters (e.g. benzoates, butanoates)
D) methods for the synthesis of amides

A) Methods for the Synthesis of Nitroxy Substituted Linkers

The nitrate esters may be prepared:

A1) By direct nitration of an alcoholic residue with concentrated nitric acid

A2) By nucleophilic substitution of an alkyl halide (preferably bromide or iodide) with silver nitrate. The latter is the preferred synthesis and it is carried out in an aprotic solvent (preferably acetonitrile), at room or reflux temperature and in the dark. The starting halide is obtained by direct halogenation of hydroxyls with known methods (e.g. PBr₃) or by radical halogenation (e.g. radicalic HBr) of alkylic groups.

B) Methods for the Synthesis of in vivo Hydrolyzable Bonds

The bond indicated by the undulated line in the compounds of general formula (I) and (II) is hydrolyzable in vivo, by metabolic or enzymatic activity, and in particular it is a carboxylic ester, a glycoside, an acetal, a ketal, a phosphoric ester, a phosphonic ester, a sulphonic ester. For the synthesis of said bonds it is possible to use several methods of synthesis known in literature. For a complete review see the above-cited “Advanced Organic Chemistry”, in particular the chemical classes index: esters and amides on page 1275 and 1281; acid halides (esters and amides precursors) on page 1269; glycosides, acetals and ketals on page 1269; phosphonic esters on page 1295; sulphonic amides and sulphonic esters on page 1296. As for the synthesis of alkyl halides (intermediates for the synthesis of nitrate esters via nucleophilic substitution) see on page 1274.

C) Methods for the Synthesis of Nitroxy Substituted Organic Esters (E.G. Benzoates, Butanoates)

The carboxylic esters are synthetized by:

C1) condensation reaction of an acid and an alcohol in the presence of a dehydrating agent

C2) acylic nucleophilic substitution reaction of an alkyl halide and an alcohol

C3) aliphatic nucleophilic substitution reaction of an aliphatic halide, preferably bromide, and a carboxylate ion

D) Methods for the Synthesis of Amides

D1) The carboxylic amides are synthetized by acylic nucleophilic substitution of an acid halide and an amine.

The association of the present invention is useful for the preparation of safe and effective medicaments mainly for primary and secondary prevention and for the treatment of coronary diseases.

Generally, the association of the invention has therapeutical applications for the prevention and treatment of several cardiovascular, inflammatory, tumoral, ischemic, neurodegenerative diseases.

The association of the invention is useful for the preparation of a medicament for the prevention and treatment of arteriosclerosis and hyperlipoproteinemie (e.g. hypercholesterolemia, hyperlipidemia), for the prevention and treatment of myocardial infarction, coronary insufficiency, peripheral arteriopathies, vasculitis, restenosis, ischemia (e.g. cardiac, cerebral, pulmonary), as anticoagulant and platelet aggregation inhibitor, for the prevention and treatment of thrombosis (e.g. intracardiac or of cerebral arteries), for the prevention and treatment of cerebrovascular diseases (e.g. cerebral stroke), cerebral ischemia and related cerebrovascular injuries, for the prevention and treatment of diabetes mellitus, for the prevention and treatment of bone diseases (e.g. osteoporosis, Paget's disease), for the prevention and treatment of dermatological diseases (e.g. diabetic ulcers, psoriasis, dermatitis), as analgesic, for the prevention and treatment of inflammatory and/or immune diseases (e.g. rheumatoid arthritis, osteoarthritis, multiple sclerosis, ulcerative colitis, graft rejection), for the prevention and treatment of neurodegenerative diseases (e.g. Alzheimer's disease, Parkinson disease, amyotrophic lateral sclerosis, Huntington's disease), for the prevention and treatment of tumors and in particular for the prevention of cancerogenesis, for inhibition of angiogenesis, for inhibition of cellullar proliferation, for inhibition of metastases, for the prevention of neoplastic cachexia.

Preferably, the association of the invention is useful for the preparation of a medicament for primary and secondary prevention and the treatment of myocardial infarction and stroke, for the prevention and treatment of diabetes mellitus, for the prevention of Alzheimer's disease and the prevention and treatment of colon-rectum tumors.

In the pharmaceutical composition of the invention, the HMG-CoA reductase inhibitor is administered in an amount from 0.05 mg/kg to 5 mg/kg, the nitrate prodrug of aspirin or salicylic acid is administered in an amount from 0.5 mg/kg to 50 mg/kg and the nitrate prodrug of vitamin E is administered in an amount from 0.1 mg/kg to 50 mg/kg.

Preferably, the HMG-CoA reductase inhibitor is administered in an amount from 0.1 mg/kg to 0.3 mg/kg, the nitrate prodrug of aspirin or salicylic acid is administered in an amount from 2 mg/kg to 10 mg/kg and the nitrate prodrug of vitamin E is administered in an amount from 1 mg/kg to 3 mg/kg.

An object of the present invention is also a method for treating patients in need of therapeutical treatment comprising the administration to said patients of:
a. an amount of a first compound, said first compound being an HMG-CoA reductase inhibitor or a pharmaceutically acceptable salt thereof
b. an amount of a second compound, said second compound being a nitrate ester or a pharmaceutically acceptable salt thereof

wherein said first compound and said second compound are each optionally and independently administered together with pharmaceutically acceptable excipients.

Preferably, in said method, the HMG-CoA reductase inhibitor is selected from atorvastatin hemicalcium salt, fluvastatin sodium salt, pitavastatin hemicalcium salt, pravastatin sodium salt, rosuvastatin hemicalcium salt and simvastatin and a nitrate prodrug of aspirin, salicylic acid or vitamin E is used as a nitrate ester.

Even more preferably, 2-acetyloxy-benzoic acid 3-nitroxymethyl-phenyl ester (Ia) is used as a nitrate ester.

The association of the present invention may be administered orally, rectally, parenterally or by local application (dermal, topical, transdermal, ocular, inhalation etc.)

The various pharmaceutical compositions may be prepared according to the known art (see for example Remington's Pharmaceutical Sciences, Mack Publishing Company, Easter, Pa., 15th Edition (1975)).

The association of the present invention may also be formulated in modified release pharmaceutical formulations which can be administered orally (diffusion systems, dissolution systems, erodable systems, osmotic systems and systems with ionic exchange resins), parenterally and by transdermic release therapeutic systems (with reservoir, with membrane and with diffusion through polymeric membrane).

Some compounds of general formula (I) and (II) are scarcely water soluble because they are very lipophilic.

In this case it is necessary to increase the solubility of said compounds by treating them with excipients which can amorphize them because in the amorphized state there is a better solubility and a better solubilization rate than in a crystallized state. In this way their bioavailabilities increase.

To obtain the compounds of general formula (I) and (II) in an amorphized state it is necessary to treat said compounds with excipients which can amorphize them. For this purpose, a process of lyophilization, kneading and preferably co-grinding or spray-drying may be used. In particular in the spray-drying technique the active principle is dissolved in a solvent, for example an alcohol, and the so obtained solution is mixed at room temperature with a solution or a suspension of excipients capable to amorphize the compounds of general formula (I) and (II). The resulting solution or suspension is treated in a spray-drying equipment. This last technique is also called solid dispersion technique and is particularly preferred. The excipients are selected from one or more of the following classes: polyalcohols (e.g. mannitol, sorbitol); monosaccharides or disaccharides (e.g. glucose, fructose, lactose); polysaccharides and their derivatives (e.g. microcrystalline cellulose, hydroxypropylmethylcellulose (HPMC), polyvinylpyrrolidone (PVP); cyclodextrins and their derivatives (e.g. α-cyclodextrin, β-cyclodextrin, methyl-β-cyclodextrin, carboxymethyl-β-cyclodextrin, acetyl-β-cyclodextrin, 2-hydroxypropyl-γ-cyclodextrin, γ-cyclodextrin, dimethyl-β-cyclodextrin, 2-hydroxyethyl-β-cyclodextrin, 2-hydroxypropyl-β-cyclodextrin, 3-hydroxypropyl-β-cyclodextrin, trimethyl-β-cyclodextrin); agents which can increase the membrane permeability (e.g. SNAC, sodium caprinate).

Preferably the excipients are selected from: polyvinylpyrrolidone, hydroxypropylmethylcellulose, lactose, microcrystalline cellulose, 2-hydroxypropyl-β-cyclodextrin, 3-hydroxypropyl-β-cyclodextrin.

The necessary amorphization degree, which can be measured by a calorimetric experiment with DSC, is at least of 5%, but preerably greater than 80%.

The w/w ratio among compounds of general formula (I) and (II) and the above cited excipients is preferably between 1:0.7 and 1:10.

The solid dispersions prepared with spray-drying technique using polyvinylpyrrolidone as excipient according to a drug/PVP ratio of 28/72 w/w are particularly preferred formulations for this invention. The compounds of general formula (I) and (II) so obtained by spray-drying have a better bioavailability and can be so administered also per os.

Since the presente invention relates to the treatment of diseases with a combination of active ingredients which may also be administered separately, the invention relates also to a kit to obtain a therapeutical effect in man comprising:
a. a therapeutically effective amount of an HMG-CoA reductase inhibitor or a pharmaceutically acceptable salt thereof together with pharmaceutically acceptable excipients in a first unit dosage form
b. a therapeutically effective amount of a nitrate ester or a pharmaceutically acceptable salt thereof together with pharmaceutically acceptable excipients in a second unit dosage form
c. container means for containing said first and second dosage forms.

Preferably, said kit contains, as an HMG-CoA reductase inhibitor, a drug selected from atorvastatin hemicalcium salt, fluvastatin sodium salt, pitavastatin hemicalcium salt, pravastatin sodium salt, rosuvastatin hemicalcium salt or simvastatin.

Preferably, said kit contains, as a nitrate ester, a nitrate prodrug of aspirin, salicylic acid or vitamin E. More preferably, said kit contains 2-acetyloxy-benzoic acid 3-nitroxymethyl-phenyl ester (Ia) as a nitrate ester

The following examples illustrate the invention without limiting the scope thereof.

### SYNTHESIS EXAMPLES

### Example 1

### paranitroxymethylbenzoic acid (ZI)

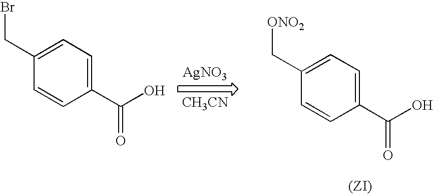

To a suspension of 5.38 g of 4-bromomethylbenzoic acid (0.025 mol) in anhydrous acetonitrile (20 ml), silver nitrate (AgNO₃) (4.95 g, 0.029 mol), dissolved in anhydrous acetonitrile (10 ml), was added drop by drop. The reaction mixture was left away from light, under stirring, at reflux (81° C.) for 8 hours and at room temperature for other 16 hours. The suspension was filtered with buchner: the precipitate, which is constituted by the product and AgBr, was dissolved in methanol (150 ml), then it was heated and finally it was filtered. The solvent was evaporated at reduced pressure and the desired product was obtained (3.75 g).

Yield: 80%. ¹H-NMR (500 MHz, DMSO-d₆): δ 7.98 (d, 2H, J=8.1 Hz), 7.58 (d, 2H, J=8.1 Hz), 5.65 (s, 2H); IR (KBr) cm⁻¹: 1690 (C═O), 1670 and 1270 (N═O).

### Example 2

### p-nitroxymethylbenzoyl-α-tocopherol (IIa)

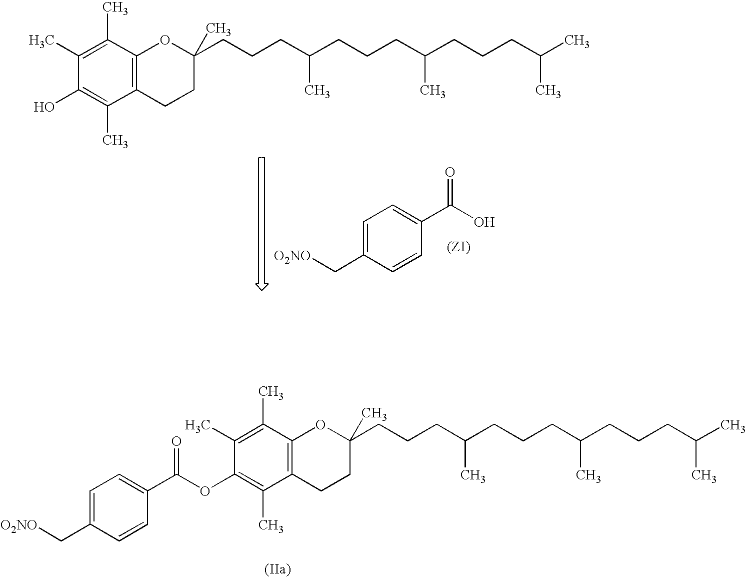

578 mg of N,N′-dicyclohexylcarbodiimide (DCC) (2.8 mmol), 607 mg (3.08 mmol) of paranitroxymethylbenzoic acid (ZI) (synthetized according to example 1) and a catalytic quantity of 4-dimethylamminopyridine (4-DMAP) were added, under nitrogen atmosphere, to a solution of α-tocopherol (vitamin E) (Sigma-Aldrich code T-3251) (1.2 g, 2.8 mmol) in CH₂Cl₂ (34 ml).

The reaction mixture is left under stirring for 5 hours. Et₂O is added to precipitate DCU which has been produced during the reaction and then the reaction is filtered upon cotton. The solution was deprived of the solvent at reduced pressure.

The crude product was purified by flash cromatography, packing the column with hexane and eluting with a mixture hexane/diisopropylic ether/CH₂Cl₂ 85/5/10.

The desired product (1.497 g) was obtained. Yield: 88%.

R_{f}=(hexane/diisopropylic ether 6:4): 0.61

¹H-NMR (CDCl₃) δ 8.27 (2H, d, J=8.53 Hz), 7.53 (2H, d, J=8.53 Hz), 5.48 (2H, s), 2.62 (2H, m), 2.11 (3H, s), 2.05 (3H, s), 2.01 (3H, s), 1.85-1.70 (2H, s broad), 1.61-1.05 (26H, CH₂ and CH aliphatic chain, CH₃ ring), 0.88-0-83 (12H, m, CH₃aliphatic chain).

¹³C-NMR (CDCl₃) δ 164.47, 149.60, 140.59, 137.78, 130.67, 128.67, 126.82, 125.04, 123.21, 117.56, 75.14, 73.66, 39.40, 37.49, 37.43, 37.42, 32.80, 27.98, 24.81, 24.45, 22.70, 22.61, 21.06, 20.64, 19.75, 19.70, 13.03, 12.17, 11.84.

MS (EI, 70 eV) m/Z 610.4 (M⁺H).

Elementary analysis:

| | | | |
|---|---|---|---|
| Calculated | C: 72.87% | H: 9.09% | N: 2.30% |
| Found | C: 72.86% | H: 9.09% | N: 2.28% |

### Example 3

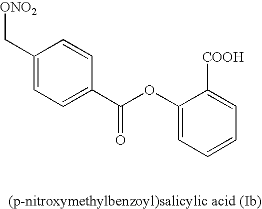

The compound is synthesized according to the synthesis described in the example 2 using, as reagents, salicylic acid (Sigma-Aldrich catalog S-7401) and paranitroxymethylbenzoic acid (ZI) synthesized according to example 1.

Yield 81%. MS (EI, 70 eV): m/z 318.05 (M⁺H)

Elementary analysis:

| | | | |
|---|---|---|---|
| Calculated | C: 56.79% | H: 3.49% | N: 4.42% |
| Found | C: 56.71% | H: 3.45% | N: 4.47% |

### PHARMACOLOGICAL EXAMPLE

Male Wistar rats (weighing 45-50 g) were housed in polycarbonate cages, 5 animals per cage, maintained at a constant temperature of 22±2° C. and at 55±15% relative humidity, with a light-darkness cycle of 12 hours, fed on “4RF21 pellet feed” (Mucedola), with tap water to drink ad libitum.

The control group consisted of 14 animals, whereas the group treated with simvastatin (Calbiochem catalog code 567020), that one treated with 2-acetyloxy-benzoic acid 3-nitroxymethyl-phenyl ester (Ia) (synthesized according to EP871606), that one treated with a mixture of both and that one treated with a mixture of simvastatin and p-nitroxymethylbenzoyl-α-tocopherol (IIa) (synthesized in the example 2) consisted of 10 animals each, according to the following experimental design:
control (no treatment)
simvastatin 140 mg/kg
(Ia) 140 mg/kg
simvastatin 70 mg/kg+(Ia) 70 mg/kg
simvastatin 70 mg/kg+(IIa) 70 mg/kg

In all groups the drugs and the mixtures were administered in a single daily dose. The duration of the treatment was 14 days

24 hours after the last treatment, the animals were anaesthetized and blood samples were taken from the sublingual vein.

The blood was centrifuged at 400 rpm for 30 min and the serum thus obtained was used to evaluate, by means of an automatic analyzer, plasma levels of glutamate oxaloacetate transaminase (GOT) (U/L), glutamate pyruvate transaminase (GPT) (U/L), creatine kinase (CK) (U/L), insulin (μU/mL), total cholesterol (TC) (mg/dL) and LDL-cholesterol (LDL-C) (mg/dL).

1. a pharmaceutical composition comprising:
a. an amount of an HMG-CoA reductase inhibitor or a pharmaceutically acceptable salt thereof
b. an amount of nitrate ester or a pharmaceutically acceptable salt thereof
c. pharmaceutically acceptable excipients.

2. A pharmaceutical composition according to claim 1 wherein the HMG-CoA reductase inhibitor is selected from the following compounds or pharmaceutically acceptable salts thereof: atorvastatin (HI), bervastatin (HIX), cerivastatin (HVIII), crilvastatin, dalvastatin, fluvastatin/fluindostatin (HVII), glenvastatin, lovastatin (HVI), mevastatin, pitavastatin (itavastatin, nisvastatin, NK-104) (HX), pravastatin (HII), rosuvastatin (S-4522/ZD-4522) (HIII), simvastatin (HV).

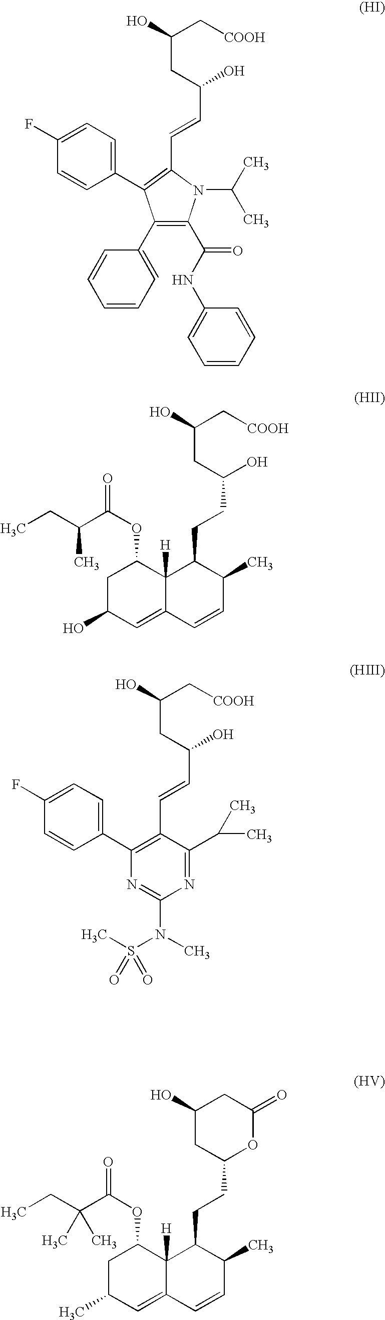

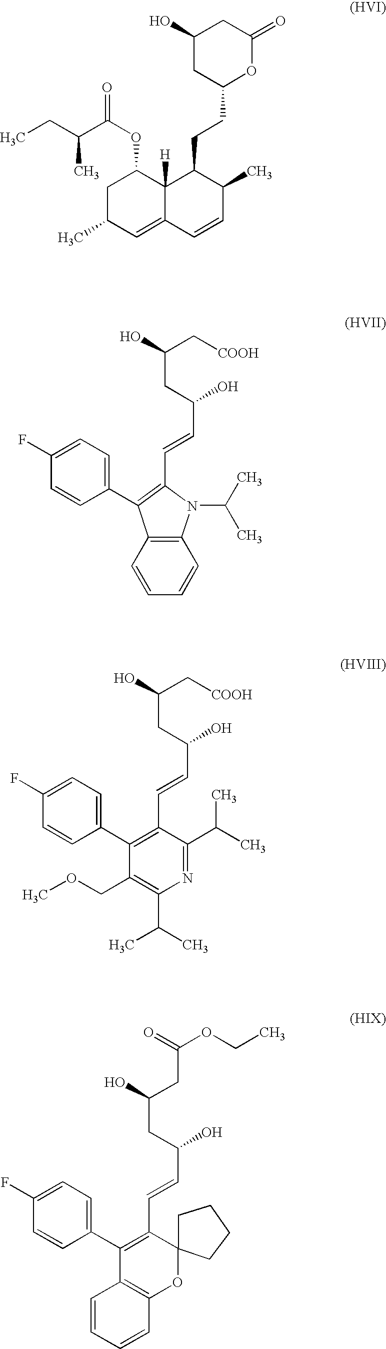

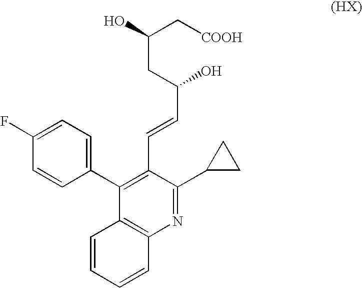

3. A pharmaceutical composition according to claim 1 wherein the HMG-CoA reductase inhibitor is selected from: atorvastatin hemicalcium salt (HIa), fluvastatin/fluindostatin sodium salt (HVIIa), pitavastatin (itavastatin, nisvastatin, NK-104) hemicalcium salt (HXa), pravastatin sodium salt (HIIa), rosuvastatin hemicalcium salt (S-4522/ZD-4522) (HIIIa), simvastatin (HV).

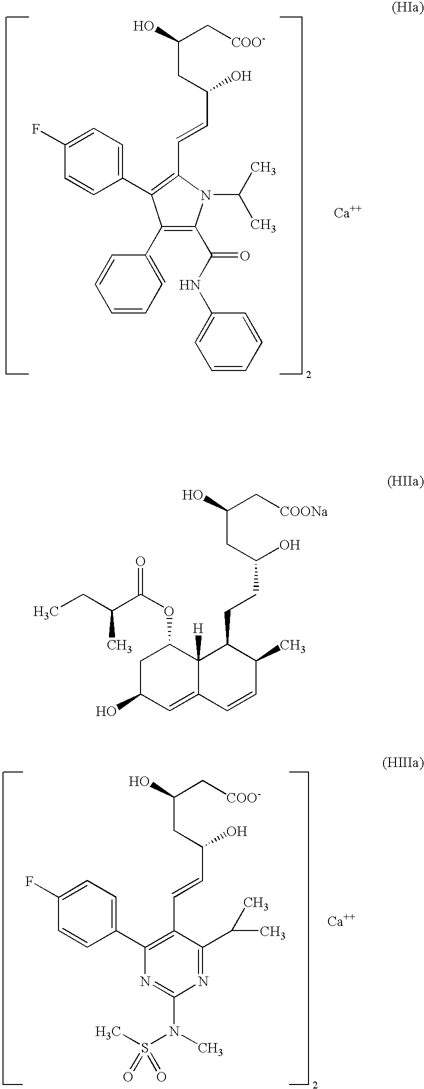

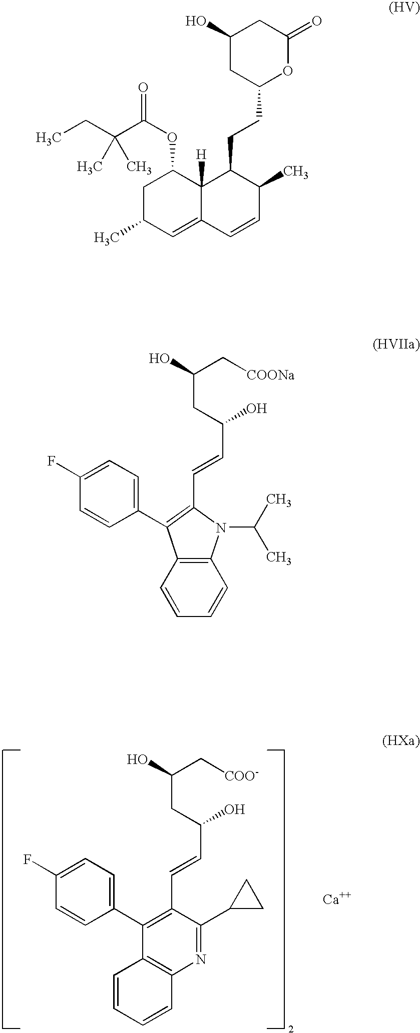

4. A pharmaceutical composition according to claim 1 wherein the nitrate ester is a nitrate prodrug of aspirin/salicylic acid or vitamin E, which are respectively represented by general formulae (I) and (II) or pharmaceutically acceptable salts thereof: 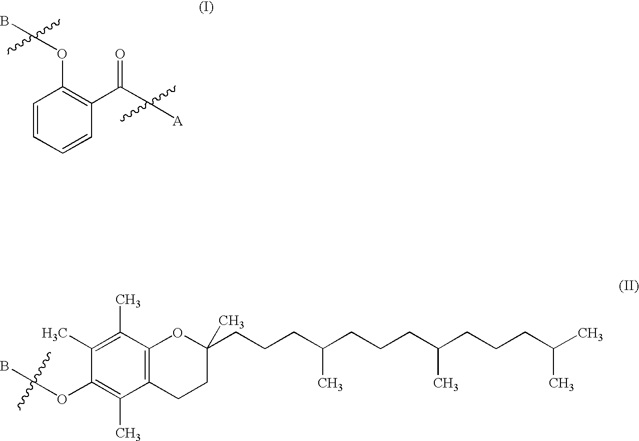
wherein:
A is selected from:
—O—R₂; —O—R₁—O—R₂; —O—R₁—S—R₂; —O—R₁—NR₂—R₂; —S—R₂; —S—R₁—O—R₂;
—S—R₁—S—R₂; —S—R₁—NR₂—R₂; —NR₂—R₂; —NR₂—R₁—O—R₂; —NR₂—R₁—S—R₂;
—NR₂—R₁—NR₂—R₂; —O—(CO)—R₂;
—O—R₁—ONO₂; —O—R₁—O—R₁—ONO₂; —O—R₁—S—R₁—ONO₂; —O—R₁—NR₂—R₁—ONO₂;
—S—R₁—ONO₂; —S—R₁—O—R₁—ONO₂; —S—R₁—S—R₁—ONO₂; —S—R₁—NR₂—R₁—ONO₂;
—NR₂—R₁—ONO₂; —NR₂—R₁—O—R₁—ONO₂; —NR₂—R₁—S—R₁—ONO₂;
—NR₂—R₁—NR₂—R₁—ONO₂; —O—(CO)—R₁—ONO₂;
—R₁— is a saturated or unsaturated, linear or branched alkylene having from 1 to 21 carbon atoms or a saturated or unsaturated, optionally heterosubstituted or branched cycloalkylene having from 3 to 7 carbon atoms or an optionally heterosubstituted arylene having from 3 to 7 carbon atoms;
—R₂ is —H or a saturated or unsaturated, linear or branched alkyl having from 1 to 21 carbon atoms or a saturated or unsaturated, optionally heterosubstituted or branched cycloalkyl having from 3 to 7 carbon atoms or an optionally heterosubstituted aryl having from 3 to 7 carbon atoms;
—R₁— and —R₂ may be substituted with —OH, —SH, —F, —Cl, —Br, —OPO₃H₂, —COOH, —NH₂ or with a saturated or unsaturated, linear or branched alkyl having from 1 to 10 carbon atoms or with a saturated or unsaturated, optionally heterosubstituted or branched cycloalkyl having from 3 to 7 carbon atoms;
—B is selected from:
—R₂; —(CO)—R₂; R₁—O—R₂; —PO(O—)O—R₂; —ONO₂; —(CO)—R₁—ONO₂;
—(CO)—R₁—O—R₁ONO₂; —(CO)—R₁—S—R₁—ONO₂;
—(CO)—R₁—NR₂—R₁—ONO₂; R₁—O—R₁—ONO₂
in the general formula (I) the derivatives wherein both -A and —B simultaneously do not contain any —ONO₂ group are excluded;
the bond indicated by the undulated line is hydrolyzable in vivo by metabolic or enzymatic activity and in particular is a carboxylic ester, a glycoside, an acetal, a ketal, a phosphoric ester, a phosphonic ester, a sulphonic ester;
the pharmaceutically acceptable salts are salts from inorganic acids, organic acids, inorganic bases or organic bases.

5. A pharmaceutical composition according to claim 4 wherein:
A is selected from —OH and the following monovalent radicals: 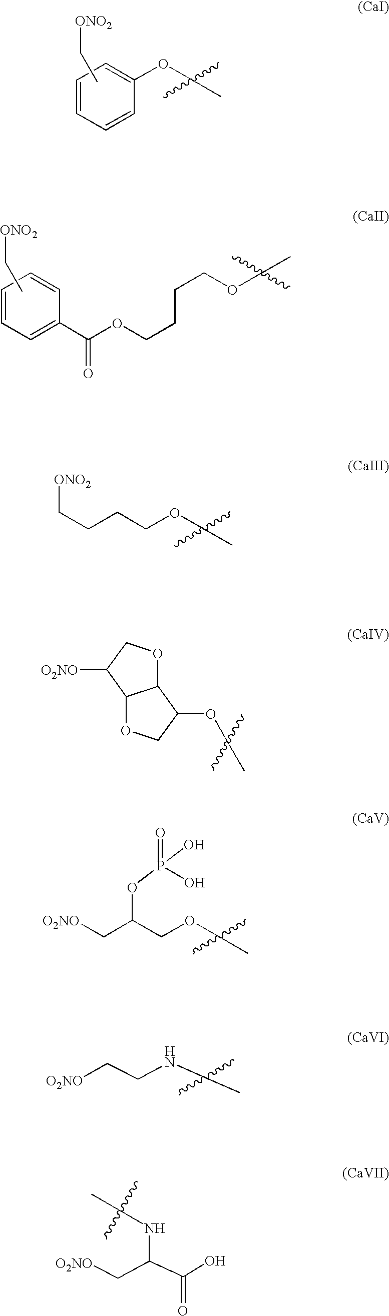 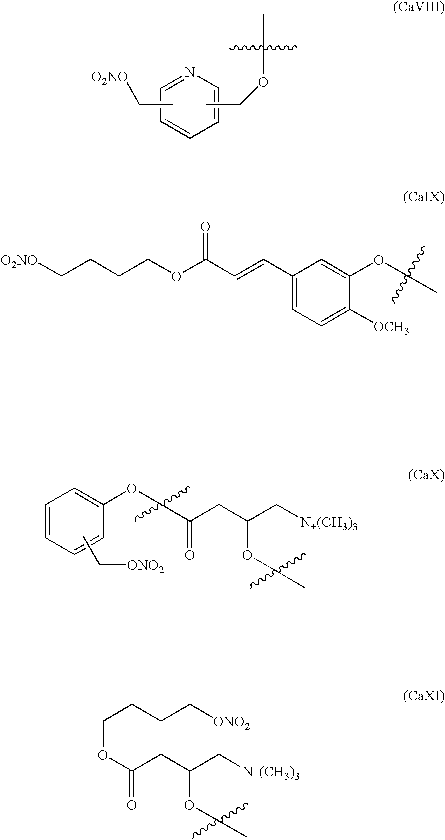
—B is selected from —H, —(CO)CH₃ and the following monovalent radicals: 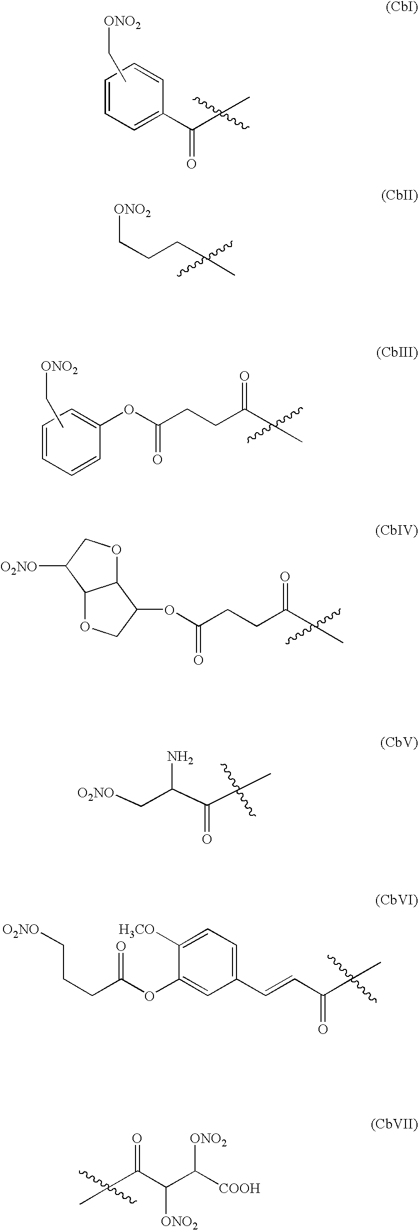 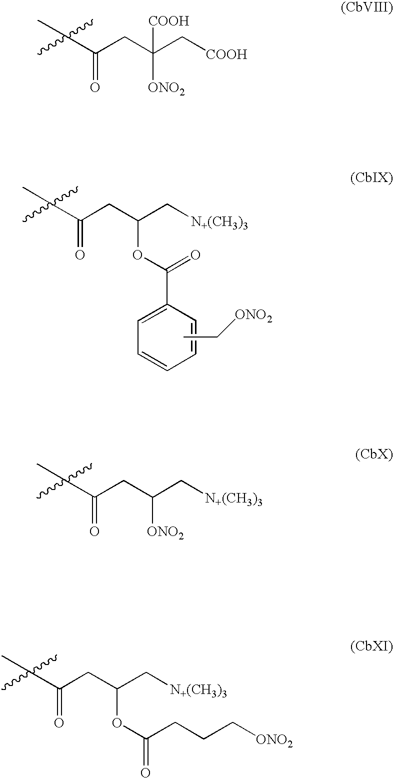
wherein the bond indicated by the undulated line may be a carboxylic ester or a carboxylic amide and wherein in the general formula (I) A may not be —OH when —B is —H or acetyl.

6. A pharmaceutical composition according to claim 4 wherein the nitrate prodrug of aspirin/salicylic acid or vitamin E is selected from the following molecules or pharmaceutically acceptable salts thereof:

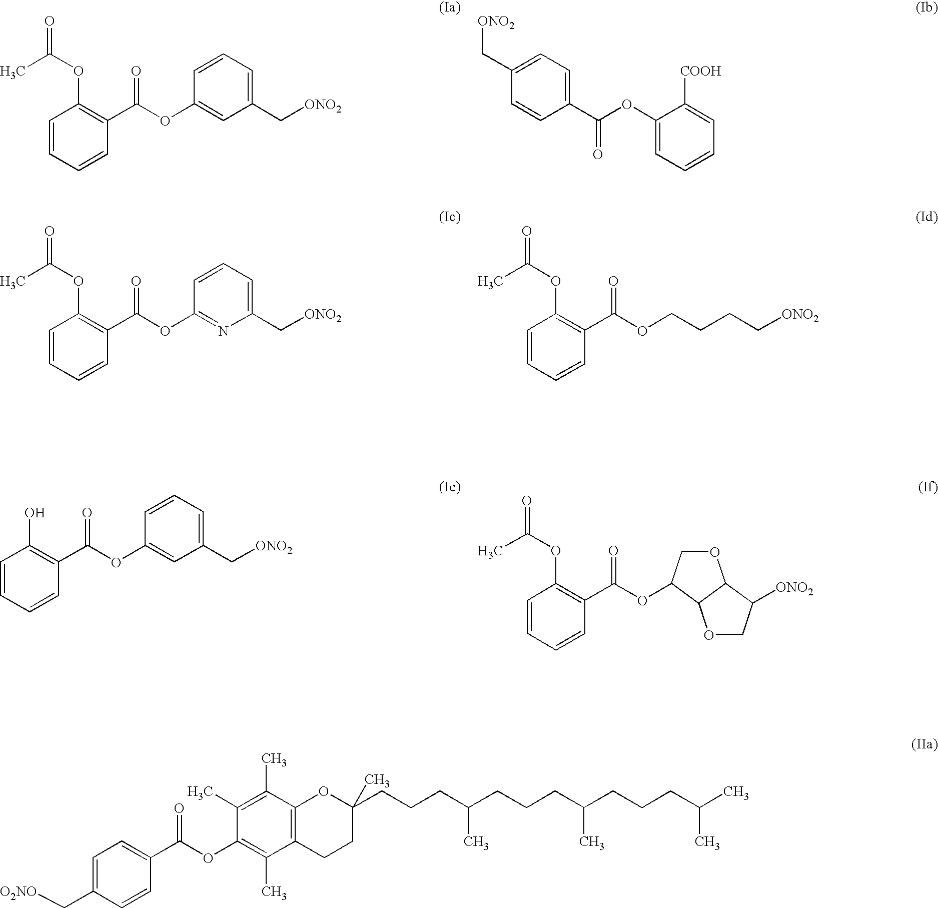

7. A pharmaceutical composition according to claim 4 wherein the nitrate prodrug of aspirin is 2-acetyloxy-benzoic acid 3-nitroxymethyl-phenyl ester (Ia)

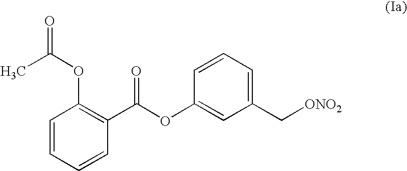

8. Use of pharmaceutical compositions according to claim 1 for the preparation of a medicament for the prevention and treatment of arteriosclerosis and hyperlipoproteinemie.

9. Use of pharmaceutical compositions according to claim 1 for the preparation of a medicament for the prevention and treatment of myocardial infarction, coronary insufficiency, peripheral arteriopathies, vasculitis, restenosis and ischemia.

10. Use of pharmaceutical compositions according to claim 1 for the preparation of a medicament as anticoagulant, as platelet aggregation inhibitor and for the prevention and treatment of thrombosis.

11. Use of pharmaceutical compositions according to claim 1 for the preparation of a medicament for the prevention and treatment of cerebrovascular diseases, cerebral ischemia and related cerebrovascular injuries.

12. Use of pharmaceutical compositions according to claim 8 for the preparation of a medicament for primary and secondary prevention and for the treatment of myocardial infarction and stroke.

13. Use of pharmaceutical compositions according to claim 8 in diabetic patients.

14. Use of pharmaceutical compositions according to claim 1 for the preparation of a medicament for the prevention and treatment of diabetes mellitus.

15. Use of pharmaceutical compositions according to claim 1 for the preparation of a medicament for the prevention and treatment of bone diseases.

16. Use of pharmaceutical compositions according to claim 1 for the preparation of a medicament for the prevention and treatment of dermatological diseases.

17. Use of pharmaceutical compositions according to claim 1 for the preparation of an analgesic medicament or a medicament for the prevention and treatment of inflammatory and/or immune diseases.

18. Use of pharmaceutical compositions according to claim 1 for the preparation of a medicament for the prevention and treatment of neurodegenerative diseases.

19. Use of pharmaceutical compositions according to claim 18 for the preparation of a medicament for the prevention of Alzheimer's disease.

20. Use of pharmaceutical compositions according to claim 1 for the preparation of a medicament for the prevention and treatment of tumors and in particular for the prevention of cancerogenesis, for inhibition of angiogenesis, for inhibition of cellullar proliferation, for inhibition of metastases, for the prevention of neoplastic cachexia.

21. Use of pharmaceutical compositions according to claim 20 for the preparation of a medicament for the prevention and treatment of colon-rectum tumors.

22. A pharmaceutical composition according to claim 1 wherein the HMG-CoA reductase inhibitor is administered in an amount from 0.05 mg/kg to 5 mg/kg, the nitrate prodrug of aspirin or salicylic acid is administered in an amount from 0.5 mg/kg to 50 mg/kg and the nitrate prodrug of vitamin E is administered in an amount from 0.1 mg/kg to 50 mg/kg.

23. A pharmaceutical composition according to claim 22 wherein the HMG-CoA reductase inhibitor is administered in an amount from 0.1 mg/kg to 0.3 mg/kg, the nitrate prodrug of aspirin or salicylic acid is administered in an amount from 2 mg/kg to 10 mg/kg and the nitrate prodrug of vitamin E is administered in an amount from 1 mg/kg to 3 mg/kg.

24. A kit for achieving a therapeutic effect in in man comprising:
a. a therapeutically effective amount of an HMG-CoA reductase inhibitor or a pharmaceutically acceptable salt thereof together with pharmaceutically acceptable excipients in a first unit dosage form
b. a therapeutically effective amount of a nitrate ester or a pharmaceutically acceptable salt thereof together with pharmaceutically acceptable excipients in a second unit dosage form
c. container means for containing said first and second dosage forms.

25. A kit according to claim 24 containing, as an HMG-CoA reductase inhibitor, a drug selected from atorvastatin hemicalcium salt, fluvastatin sodium salt, pitavastatin hemicalcium salt, pravastatin sodium salt, rosuvastatin hemicalcium salt or simvastatin.

26. A kit according to claim 24 containing, as a nitrate ester, a nitrate prodrug of aspirin, salicylic acid or vitamin E.

27. A kit according to claim 24 containing, as a nitrate ester, 2-acetyloxy-benzoic acid 3-nitroxymethyl-phenyl ester (Ia).

28. A method for treating patients in need of therapeutical treatment comprising the administration to said patients of:
a. an amount of a first compound, said first compound being an HMG-CoA reductase inhibitor or a pharmaceutically acceptable salt thereof
b. an amount of a second compound, said second compound being a nitrate ester or a pharmaceutically acceptable salt thereof
wherein said first compound and said second compound are each optionally and independently administered together with pharmaceutically acceptable excipients.

29. A method according to claim 28 wherein the HMG-CoA reductase inhibitor is selected from atorvastatin hemicalcium salt, fluvastatin sodium salt, pitavastatin hemicalcium salt, pravastatin sodium salt, rosuvastatin hemicalcium salt and simvastatin and a nitrate prodrug of aspirin, salicylic acid or vitamin E is used as a nitrate ester.

30. A method according to claim 29 wherein the nitrate ester is 2-acetyloxy-benzoic acid 3-nitroxymethyl-phenyl ester (Ia).
